(19) United States
(12) Patent Application Publication
(10) Pub. No.: US 2008/0095838 A1
(43) Pub. Date: Apr. 24, 2008

(54) Solid pharmaceutical composition containing a lipophilic active principle and preparation method thereof

(75) Inventor: Marie-Line Abou Chacra-Vernet, Nice (FR)

Correspondence Address: DICKINSON WRIGHT PLLC, 1901 L. STREET NW, SUITE 800, WASHINGTON, DC, 20036, US

(73) Assignee: CLL Pharma, Nice (FR)

(21) Appl. No.: 10/519,166

(22) PCT Filed: Jun. 24, 2003
(86) PCT No.: PCT/FR03/01933

§ 371 Date: Oct. 26, 2005

(30) Foreign Application Priority Data
Jun. 25, 2002 (FR) 02/07831

(51) Int. Cl. A61K 9/16; A61K 47/00; A61K 47/12; A61K 9/48; A61K 9/20; A61K 47/30

(52) U.S. Cl. 424/452; 424/465; 424/501; 514/772.6; 514/784; 514/788

(57) ABSTRACT

The invention relates to an orally-administered, solid pharmaceutical composition comprising, in a single same phase, a lipophilic active principle, a surface active agent, a cationic polymer which is insoluble in water at pH greater than or equal to 5 and a mineral or organic acid. The invention also relates to the preparation method of same.

The present invention relates to a solid pharmaceutical composition intended to be administered orally, comprising, within one and the same phase, a lipophilic active principle, a surfactant, a cationic polymer insoluble in water at neutral or alkaline pH and an inorganic or organic acid, and also to the method for preparing it.

Such a composition makes it possible in particular to confer on this type of active principle, when it is absorbed orally, a significantly improved bioavailability due to a dissolution that is promoted in aqueous medium.

A large number of lipophilic active principles exist which exhibit low water-solubility and for which the formulation, for oral administration, poses a certain number of major problems, in particular in terms of bioavailability.

Among these lipophilic active principles are especially the family of blood lipid-reducing agents to which fibrates, such as fenofibrate (INN), in particular belong.

Fenofibrate was placed on the market for the first time in France in 1975, in the form of gelatin capsules containing a dose of 100 mg of active principle (Lipanthyl® 100), and with a dosage of 3 or 4 gelatin capsules per day to be taken during the various meals, therefore corresponding to a daily administration of 300 to 400 mg of active principle. This pharmaceutical specialty product is still sold, to this day, in France, but under the name Sécalip® 100.

Since this first placing on the market, formulators have continually sought to improve the formulation of the lipophilic active principles, and in particular that of the fenofibrate.

In 1986, a second dosage, intended to give patients requiring only 300 mg of fenofibrate per day an opportunity for stricter adherence to the treatment, was marketed in the form of gelatin capsules containing 300 mg of active principle (Lipanthyl® 300), the dosage then being a single gelatin capsule per day. Such a dosage is made possible by the long-lasting action of fenofibrate, the elimination half-life of which is in fact approximately 20 hours. This pharmaceutically specialty product is still available in France, under the name Sécalip® 300.

However, this pharmaceutical form results in poor bioavailability of the active principle (Desager et al., J. Clin. Pharmacol., 1978, 26, 570-574; Weil et al., Drug Metabolism, 1980, 18, 115-120); Strolin Benedetti et al., Acta Pharmacol. Toxicol., 1986, 59, suppl. 5, 1967). In fact, due to its poor water-solubility, fenofibrate, and more generally lipophilic active principles, which are not very soluble at all, or even insoluble, in water, are poorly absorbed in the digestive tract and consequently exhibit incomplete bioavailability.

To improve the dissolution of fenofibrate and its bioavailability and thus to reduce the dose which must be administered, patent application FR-A-2 627 696 proposes in particular a pharmaceutical composition in which the fenofibrate is used in the form of a powder comicronized with a solid surfactant, for example sodium lauryl sulfate. The comicronized fenofibrate powder thus obtained is mixed with conventional excipients such as lactose, starch, polyvinylpyrrolidone (PVP) and magnesium stearate, and then placed in gelatin capsules.

That patent teaches in particular that the comicronization of fenofibrate with a solid surfactant makes it possible to significantly improve the bioavailability of the fenofibrate, to a greater extent than the improvement that would be obtained either by addition of a surfactant, or by micronizing only the fenofibrate, or alternatively by intimately mixing the fenofibrate and the surfactant that had been micronized separately. This method results in a novel pharmaceutical form in which the active product, comicronized with a solid surfactant, exhibits improved dissolution of the fenofibrate, and therefore an increased bioavailability, which allows, at equal effectiveness, a decrease in the daily dose of medicinal product: respectively 67 g (Lipanthyl® 67 M or Tricor®) and 200 mg (Lipanthyl® 200 M or Tricor®) instead of 100 mg and 300 mg. Thus, the patient was able to have available a pharmaceutical form needing to be taken only once a day, and which produces an effect identical to that obtained with multiple intakes. However, the method of preparation according to that patent is not entirely satisfactory insofar as it does not result in complete dissolution and bioavailability of the active principle.

Patent application EP 0 793 958 describes a method of preparing a pharmaceutical composition using fenofibrate, a surfactant and PVP. According to that document, the fenofibrate particles are mixed with a solution of PVP. The mixture obtained is then granulated with a solution of one or more surfactants. The granule thus produced is then dried.

Patent application FR-A-2 783 421 shows that it is also possible to improve the bioavailability of fenofibrate by means of a method of preparation consisting in micronizing the fenofibrate and then in granulating it in the presence of a liquid medium comprising a surfactant, water and a water-miscible alcohol and, finally, in drying the granule thus obtained.

According to patent application EP 1 048 295, the dissolution of a composition of this active principle can also be improved when the comicronization of the fenofibrate is carried out with a solid excipient other than a surfactant, such as lactose, microcrystalline cellulose or starch.

Similarly, international application WO 02/11699 describes a pharmaceutical formulation comprising micronized fenofibrate, intimately mixed with inert excipients such as lactose and microcrystalline cellulose, in which the size of the particles (less than 50 μm) is selected so as to prevent aggregation of the fenofibrate particles and to optimize their dispersion. Thus, the dissolution of the fenofibrate is improved and the bioavailability of the composition thus prepared is greater than that of the pharmaceutical specialty product Tricor® containing a dose of 200 mg and 67 mg. However, the dissolution of fenofibrate at 30 minutes is not close to 100%.

U.S. Pat. No. 6,180,138 describes a method for preparing a solid formulation of agent for regulating lipids, including fenofibrate, the dissolution and absorption characteristics of which are improved. The method described in that patent consists of a mixture of fenofibrate comicronized with, optionally, one or more excipients such as lactose, which is suspended in a solution of surfactant. An electrolyte of the sodium chloride type can be added to the solution of surfactant so as to prevent agglomeration of the fenofibrate particles by electrostatic force. The suspension is then dried by atomizing or on a fluidized air bed and, optionally, granulated and formulated into gelatin capsules or tablets.

U.S. Pat. No. 6,368,622 relates to another method for preparing a solid fenofibrate formula, consisting in melting and mixing fenofibrate and a surfactant with, optionally, another excipient. The mixture, which has been frozen, ground and reduced to powder or small granules, is then placed in gelatin capsules. This method makes it possible to obtain very small particles of fenofibrate so as to increase the specific surface area and therefore the dissolution. The dissolution results show an equivalence between the pharmaceutical compositions obtained according to the method in that patent and the specialty product Lipanthyl® 67 M.

U.S. Pat. No. 6,368,620 concerns a method for preparing a pharmaceutical formulation comprising an agent for regulating lipid metabolism, including fenofibrate, dissolved in supercritical CO₂ and for which the solution is sprayed through a nozzle so as to form small particles (nanoparticles or nanocrystals) that are readily dissolved. The addition of a surface-coating agent, before or after the nanoparticle formulation step, makes it possible to prevent agglomeration of the particles with one another. A suspension is then formed so as to collect the particles and to dry them. The dissolution of the pharmaceutical formulations of the nanoparticles or nanocrystals thus generated is compared with that of Lipanthyl®.

Another method for improving the dissolution of fenofibrate is described in patent application EP 0 904 781. It is a method for preparing fenofibrate granules from a solid dispersion, which method comprises the mixing of a solid disintegrating agent with molten fenofibrate, the cooling and the solidification of the mixture and the production of granules by sieving. The disintegrating agents used are polymers such as starch, sodium croscarmellose, sodium starch glycolate and crosslinked PVP (crospovidone). The granulated material is then introduced into the pharmaceutical compositions. However, a disintegrating agent such as crospovidone does not dissolve evenly in the molten fenofibrate and exhibits phase compatibility problems. These phenomena were demonstrated by M. T. Sheu et al., Int. J. Pharm. 1994, 103 (2), 137-146, using differential calorimetric measurements. The latter authors also discovered that fenofibrate is incompatible with PVP. Thus, the preparation of mixtures of pharmaceutical material by melting followed by solidification results in an uneven distribution and composition in the final granulated material which can lead to adverse effects on the bioavailability of the active principle. In addition, the “co-melt” method requires the use of specific equipment.

Patent application FR-A-2 722 984 describes a solid dispersion of active principles that ensures better solubility in aqueous media and better resorption in the digestive tract. The solid dispersion is prepared by dissolving the active principle in a volatile organic solvent containing a very hydrophilic polymer such as a cyclic amide, and optionally a surfactant, and then by evaporating the organic solution to dryness. The co-precipitate of the active principle and of the polymer thus formed is then ground, sieved and diluted with a pharmaceutically acceptable excipient or vehicle, or else used as it is. It is a composition comprising a surfactant, and an active principle dispersed in a hydrophilic excipient of the pyrrolidone type.

International application WO 00/72829 describes a pharmaceutical composition comprising a lipid regulating agent, including fenofibrate, and an excipient. In that pharmaceutical composition, the fenofibrate and said excipient, for example succinic acid or polyethylene glycol (PEG), form a eutectic mixture. However, these solid dispersions do not exhibit a dissolution profile close to 100% and require specific equipment in order to prepare them.

Patent application FR-A-2 758 459 describes a solid pharmaceutical composition of fenofibrate having a high bioavailability and also the method for preparing it. That document describes in particular an immediate-release fenofibrate composition comprising a water-soluble inert support coated with a layer of micronized fenofibrate, the particle size of which is less than 20 μm, of a hydrophilic polymer such as PVP and, optionally, of a surfactant. The microgranule then comprises an additional external phase or external coating, such as, for example, crosslinked PVP. The composition thus prepared shows a significant increase in the dissolution of the fenofibrate. Even though the increase in the rate of dissolution in vitro does not directly signify an increase in bioavailability in vivo, the teaching of that document lies in the use of a hydrophilic polymer and of a water-soluble inert support making it possible to organize the fenofibrate particles around a support so as to offer a greater specific surface area to the dissolution medium. In this situation, the dissolution profiles are close to 100% since the following dissolution is obtained for the modified-release tablet containing a dose of 160 mg: 83% of fenofibrate dissolved in 15 minutes and 96% in 30 minutes against, respectively, 16.5% and 55% for Lipanthyl® 200 M. This new pharmaceutical composition is currently marketed in France under the name Lipanthyl® 160 mg. However, the method of implementation is complex and expensive and requires specific equipment.

There exists therefore a need for improving the dissolution and the bioavailability of lipophilic principles which are not very soluble at all, or even insoluble, in water, and in particular that of blood lipid-reducing agents such as fenofibrate, in solid form, in order to achieve, in very short periods of time, a level close to 100%, in any case greater than 80% in 30 minutes, without having to resort to complex and expensive methods such as, for example, the preparation of water-soluble microgranules or of solid dispersions.

The applicant has demonstrated, surprisingly, that it is possible to solve this problem by means of a single-phase solid pharmaceutical composition comprising a solid and micronized lipophilic active principle, a surfactant, a cationic polymer insoluble in water at pH greater than or equal to 5, for example of the polymethacrylate type, and an organic or inorganic acid.

The present invention also relates to the method for preparing such a composition.

Pharmaceutical compositions based on lipophilic active principles comprising polymethacrylates are known. These formulations are generally used to delay or control the release of the active principle in the organism.

International application WO 82/01649 describes a formulation of microgranules based on fenofibrate comprising a neutral core consisting of starch and of sucrose. The neutral core is coated with a solution of fenofibrate with, optionally, one or more excipients, and with a microporous second layer consisting of a polymer that is compatible with oral administration, in particular gum arabic, cellulose ethers and esters, polymethacrylates, etc. This formulation makes it possible to produce a medicinal product having a delayed effect controlled by the mixture of granules having various rates of release of fenofibrate. For example, the neutral granules not coated with polymer make it possible to obtain a predetermined concentration of fenofibrate.

Patent application FR-A-2 602 423 relates to a pharmaceutical composition comprising controlled-release fenofibrate granules. Each granule consists of an inert core, of a layer of fenofibrate and of a protective layer based on methacrylic polymers, on PVP, on cellulose derivatives or on polyethylene glycols. In the base layer, the fenofibrate is present in the form of microcrystalline particles less than or equal to 50 microns (μm) in size. The use of such crystals makes it possible to obtain an even absorption of the fenofibrate, contrary to that which is obtained by following the teaching of international application WO 82/01649.

International application WO 96/36318 relates to a 3-phase pharmaceutical form for constant and slow release of an amorphous active ingredient for single daily administration. The first phase consists of a core containing in particular amorphous fenofibrate, PVP, a cellulose ether as excipient and crystallization inhibitor, and a surfactant which improves the solubilization and the absorption of the amorphous active principle in the gastrointestinal tract. The second phase contains a cellulose ether and a mixture of mono-, di- and triglycerides so as to modify the kinetics of release of the active principle. The third phase consists of a weakly soluble coating film consisting of gastro-resistant polymer, in particular Eudragit® L100-55, which is a copolymer of methacrylic acid and of ethyl acrylate (noncationic polymer).

Patent application FR-A-2 772 615 concerns a multilayer (bilayer) tablet for the immediate and then prolonged release of active substances, comprising at least two superimposed layers. The first layer allows immediate release of the active principle. The second layer consists of an inert porous polymeric matrix that is non-biodegradable, in which is dispersed a second active substance, which may be identical to that present in the first layer. The active substances may in particular be blood lipid-reducing agents and the polymeric matrix is, for example, a noncationic copolymer of methacrylic acid and of ethyl acrylate or else a cationic copolymer such as a copolymer of ethylammonium methacrylate and of ethyl methacrylate (Eudragit® RL, RS, L). This second matricial layer is responsible for the controlled release of the active principle.

Finally, international application WO 00/72825 relates to a solid formulation comprising fenofibrate or a statin dispersed in an amorphous hydrophilic polymer. The fenofibrate is present in the formulation in an amorphous metastable form. Designated among the amorphous polymers are, inter alia, the polymethacrylates of the trade mark Eudragit®. According to that document, it is hoped that there will be an improvement in dissolution since the active principles and the polymethacrylates are in an amorphous form. However, nothing demonstrates an improvement in dissolution as it is stated by the authors, all the more so since the results reported demonstrate a loss of bioavailability and a delayed effect.

All these documents demonstrate that the Eudragit®-type polymers are generally used as a coating film in order to delay the release of an active principle. Thus, the dissolution values cannot be greater than 80% in 30 minutes.

According to K. Lehmann et al., “Acrylic polymers: A review of pharmaceutical applications”, STP Pharma Sciences, 1997, 403-437, polymethacrylic polymers or copolymers, in particular the Eudragit®-type polymers, can be used in pharmaceutical forms for acting on the release of active principles in the gastrointestinal tract:
either immediately,
or with a lag-time,
or in a prolonged manner.

The main acrylic polymers are the carbomers (Carbopol®), and the copolymers of trimethylammonioethyl chloride/methacrylate (Eudragit® RS/RL), of methacrylic acid/methyl methacrylate (Eudragit® L100, S100, L100-55), and of poly(dimethylaminoethyl methacrylate), of methyl methacrylate and of butyl methacrylate (Eudragit® E100).

According to patent application EP 0 436 370, it is possible to prepare “pulsed”-release formulations characterized by a lag-time, comprising a core of active principle and an organic acid, in particular succinic acid, and a coating film based on Eudragit® RS or RL having a quaternary ammonium group. Since the coating film is slightly permeable to water, the pharmaceutical substance is dissolved or released only after a predetermined period of time (depending on the thickness of the coating), but when the digestive juice has gradually penetrated into the preparation and thus dissolved the organic acid, the permeability of the polymer is increased, hence a rapid dissolution and release of the active principle. The probable mechanism of such a unique scheme for dissolution and release of the active principle could be explained by the fact that the dissolved organic acid reacts with the ethyltriethylammonium group contained in the coating film formed by the aqueous dispersion and that, thus, the very slightly water-permeable polymer is converted into a water-permeable polymer.

The Eudragit®-type polymers comprising a tertiary amine group, in particular Eudragit® E, are mainly used as a film for coating the final pharmaceutical forms so as to mask the taste thereof. These films are insoluble under the virtually neutral pH conditions (pH 5 to 7) of the oral environment, but are very soluble in an acid environment such as the stomach. They dissolve rapidly at pH less than 5 due to a high concentration of tertiary amine groups of the N,N-dimethylaminoethyl methacrylate monomer (50% by weight of the polymer). On the other hand, this polymer swells at a pH above 5 due to the very hydrophobic amine groups. This polymer can also be used in slow-release formulations. In fact, according to P. Lis et al., Drug. Dev. Ind. Pharm., 1989, 15, 1999-2016, when Eudragit® E coats theophylline granules, the release kinetics are zero order kinetics.

In rare cases, Eudragit® E can provide immediate-release pharmaceutical forms. According to H. Suzuki et al., Chem. Pharm. Bull., 1996, 44, 364-371, benidipine hydrochloride, which is a calcium antagonist, is slightly soluble in a weak acid, to such an extent that its bioavailability is influenced by the variability in the gastric acidity of patients. On the other hand, the molecule is relatively insoluble in the organic solvents generally used in solid dispersion preparations. According to the solvent used, the authors have shown that Eudragit® E is capable of solubilizing benidipine hydrochloride by means of molecular interaction between the active principle and the film. Thus, it has been possible to prepare solid dispersions of benidipine, which have shown an improved rate of dissolution (pH 6). However, the benidipine in hydrochloride form is an ionized species which does not behave like fenofibrate. Since fenofibrate is not a salt, there is no direct ionic interaction possible with the Eudragit® film.

Thus, a subject of the present invention is a solid pharmaceutical composition intended to be administered orally, characterized in that it comprises, within one and the same phase (internal phase):
at least one solid and micronized lipophilic active principle,
at least one surfactant,
at least one cationic polymer insoluble in water at pH greater than or equal to 5, and
at least one organic or inorganic acid.

Such a pharmaceutical composition is not in the form of a multilayer tablet but in the form of a homogeneous composition consisting of a single phase within which all the ingredients described above are intimately mixed. In addition, the insoluble cationic polymer does not confer any delayed effect on the pharmaceutical composition.

According to the present invention, the term “lipophilic active principle” covers any lipophilic substance that is not very soluble at all, or even insoluble, in water and that has a pharmacological, therapeutic, etc., activity.

The expression “not very soluble at all” in water is intended to mean, according to the invention, a solubility of less than or equal to 0.1 mg/ml or an octanol/water partition coefficient (P), expressed by log P, of greater than 2 and preferably greater than or equal to 4. In fact, the lipophilicity of an active principle (AP) can be determined as a function of its partition coefficient (P) between octanol and water, which corresponds to the ratio of the concentration of the AP in octanol (C_{Oct})/concentration of the AP in water (C_{water}). When the ratio P is greater than 1, this means that C_{Oct} is greater than C_{water} and that, consequently, the AP is lipophilic (log P>0). It can therefore be deduced therefrom that the higher the log P of an AP, the more pronounced the lipophilic nature thereof. These active principles are, in practice, in class 2 and class 4 of the biopharmaceutical classification proposed in 1995 by Amidon et al., Pharm. Res., 1995, 12, 413-420.

These lipophilic active principles can in particular be chosen from blood lipid-reducing agents, steroid hormones, antifungal agents, retinoids, steroidal anti-inflammatories, nonsteroidal anti-inflammatories (NSAIDs), antiretroviral agents, protease inhibitors (“navirs”), antacids, proton pump inhibitors, antiemetics, liposoluble vitamins, cardiovascular system drugs, anti-platelet aggregation agents, anticancer agents, certain plant extracts and their isolated or derived active principles, immunosuppressants, central nervous system drugs, antimigraine agents, antibiotics and antiparasitic agents.

Among the blood lipid-reducing agents, mention may in particular be made of fibrates, such as 2-(4-(4-chlorobenzoyl)phenoxy)-2-methylpropanoic acid 1-methylethyl ester, also called fenofibrate, and products belonging to the fibrate class, such as bezafibrate, ciprofibrate and gemfibrozil.

Among the other blood lipid-reducing agents, mention may also be made of bisthioethers, including probucol and tiadenol, the class of HMG Co-A reductase inhibitors (statins), such as, for example, simvastatin, mevastatin, lovastatin, atorvastatin, pravastatin, fluvastatin, cerivastatin, and also the class of ACAT inhibitors, such as melinamide and its structural analogues.

As steroid hormones, mention may in particular be made of derived estrogens and esters of estradiol, progesterone, danazol, testosterone and testosterone esters and derivatives. Mention may also be made of anti-androgens, including flutamide and nilutamide; 5α-reductase inhibitors, competitive inhibitors of testosterone, such a finasteride; quinazoline derivatives such as alfuzosine; nonsteroidal agonists/antagonists of estrogen receptors, such as tamoxifen and raloxifen.

Among the antifungal agents, mention may in particular be made of azole-containing antifungal agents (conazoles), including itraconazole, miconazole, ketoconazole and fluconazole, and also griseofulvin, amphotericin B and terbinafine.

Among the retinoids, mention may be made, firstly, of retinoids derived from vitamin A, such as tretinoin, also known as all-trans retinoic acid or as all-trans vitamin A acid, isotretinoin which corresponds to the 13-cis isomer of tretinoin and which, as a result, is also called 13-cis retinoic acid or 13-cis vitamin A acid, 9-cis retinoic acid or 9-cis vitamin A acid, acitretin, etretinate, but also acetylenic retinoids such as tazarotene, retinoids derived from naphthalene, such as lonapalene and 2-(5,6,+,8-tetrahydromethyl-2-anthryl)-4-thiophenocarboxylic acid, and retinoids containing an adamantyl ring, such as adapalene, 6-[3-(1-adamantyl)-4-hydroxyphenyl]-2-naphthoic acid and 4-[3-(1-adamantyl)-4-methoxybenzamido]benzoic acid and their esters.

As steroidal anti-inflammatories, mention may in particular be made of glucocorticoids such prednisolone, cortisone and its esters and derivatives.

As NSAIDs, mention may in particular be made of mefenamic acid, naproxene, nabumetone, ibuprofen and COX-2 inhibitors such as celecoxib, rofecoxib, parecoxib and valdecoxib.

The antiretroviral agents and the protease inhibitors are compounds that are not very soluble in water at all, the partition coefficients of which can be calculated or determined analytically, among which mention may be made of amprenavir (solubility 0.04 mg/l), saquinavir and saquinavir mesylate (solubility 2.22 mg/ml) and ritonavir (virtually insoluble in water).

As antacids and proton pump inhibitors, mention may in particular be made of omeprazole, pantoprazole, rabeprazole (or pariprazole), lansoprazole and timoprazole.

As antiemetics, mention may in particular be made of domperidone, and the serotonin antagonists (“setrons”) such as ondansetron, granisetron and azasetron.

As liposoluble vitamins, mention may in particular be made of vitamins A or retinol, D including calcitriol, E or tocopherols, and K or menadione.

Among the cardiovascular system drugs, mention may be in particular be made of angiotensin II antagonists (sartans) such as valsartan, losartan, irbesartan, candesartan, tasosartan, telmisartan; α- and β-blockers such as carvedilol, celiprolol; calcium inhibitors (dihydropyridines) such as verapamil, diltiazem, nifedipine and nitrendipine. Mention may also be made of other compounds; antihypertensives, such as renin-inhibiting peptides, oxazolidinone derivatives or glycol peptides substituted with amino residues and/or azole-containing or thiazole-containing heterocyclic rings.

As antiplatelet aggregation agents, mention may in particular be made of ticlopidine and clopidogrel hydrogen sulfate; coumarin anticoagulants including warfarin and compounds of the indandione group, including phenylindandione.

As anticancer agents, mention may in particular be made of paclitaxel and docetaxel, which are water-insoluble compounds; extracts and alkaloids of Vinca minor, such as vincristine, vincaleukoblastine or vinblastine, vincamine and their derivatives; alkaloids of Ochrosia elliptica, including ellipticin.

Among the plant extracts and their isolated or derived active principles, mention may in particular be made of alkaloids such as yohimbine, flavonoids including diosmin, rutin and its derivatives such as troxerutin; extracts of Pygeum africanum or of Serenoa repens.

As immunosuppressants, mention may in particular be made of cyclosporin and tacrolimus.

Among the various central nervous system drugs, are tranquilizers, sedatives, hypnotics and anesthetics. By way of example, mention may be made of barbiturates such as thiobarbiturates; anxiolytic drugs such as benzodiazepines; antihistamines such as terfenadine, loratadine, desloratadine and cetirizine; tricyclic and serotoninergic antidepressants such as fluoxetine, paroxetine, sertraline and citalopram.

Among the antimigraine agents, mention may be made of compounds of the serotoninergic “triptan” group, such as oxitriptan, sumatriptan and almotriptan.

Among the antibiotics, mention may in particular be made of third-generation cephalosporins such as cefixime trihydrate and cefpodoxime proxetil; macrolides such as azithromycin, clarithromycin, roxithromycin, josamycin, spiramycin; synergistins such as pristinamycin; quinolones and quinoxalines, including carbadox.

Among the antiparasitic agents, mention may be made of antimalarials such as halofantrine, mefloquine, proguanil, pyrimethamine, extracts of Artemisia spp and the substances isolated from these extracts and their derivatives such as artemisin, artemisinin; the avermectin series, including the virtually water-insoluble ivermectin; the anthelmintics derived from benzimidazole for veterinary use, such as, for example, tiabendazole, albendazole, mebendazole, fenbendazole and triclabendazole; the class of salicylanilides for veterinary use, used in fasciolosis (flukicides) and other forms of parasitosis, comprising in particular bromoxanide, brotianide, clioxanide, closantel, oxyclozanide, rafoxanide and also dibromosalan and tribromosalan.

According to the invention, the active principle(s) is (are) preferably chosen from blood lipid-lowering agents, among which fenofibrate is most particularly preferred, steroid hormones, among which progesterone is most particularly preferred, and antifungal agents, among which itraconazole is most particularly preferred.

The active principle(s) preferably represent(s) from 10 to 90% by weight of the total weight of the pharmaceutical composition, these percentages being, quite obviously, modulated according to the needs of the formulation with regard to the effective doses of the active principle under consideration.

According to an advantageous embodiment of the invention, the micronized active principle is present, before it is mixed with the other constituents of the present pharmaceutical composition, in the form of a powder, the particles of which are homogeneous in size and less than or equal to 45 μm and, if necessary, less than or equal to 10 μm.

As was seen above, the presence of a surfactant is one of the essential characteristics of the pharmaceutical composition in accordance with the invention.

This surfactant is preferably chosen from compounds having a high hydrophilic/lipophilic balance (HLB) value preferably greater than or equal to 15. Among such surfactants, mention may in particular be made of sodium lauryl sulfate (HLB 40), poloxamers (HLB 16-29), macrogol ethers of organic alcohols (HLB 15-18), and sucrose esters of organic acids (HLB 15-16).

According to a preferred embodiment of the invention, the surfactant is sodium lauryl sulfate.

The surfactant(s) that can be used in the pharmaceutical composition in accordance with the invention preferably represents from 1 to 10% by weight of the total weight of said composition, and even more preferably from 5 to 10% by weight.

According to a particular embodiment of the invention, the surfactant is in solid form. In this case, it can be comicronized with the active principle.

The techniques for micronizing active principles (in the presence or absence of a solid surfactant) are well known to those skilled in the art and can in particular be carried out by means of air-jet grinders or of liquid-jet grinders (Microfluidizer®). An active principle comicronized with sodium lauryl sulfate can in particular be used.

The active principle/surfactant ratio by weight is preferably between 100/1 and 5/1.

Among the cationic polymers insoluble in water at pH greater than or equal to 5 that can be used in accordance with the invention, mention may be made of acrylic polymers comprising a tertiary amine group, such as polymers of aminoalkyl methacrylate type, soluble in acid medium, i.e. at pH below 5. Among these polymers, mention may, for example, be made of the terpolymer of poly(dimethylaminoethyl methacrylate), of methyl methacrylate and of butyl methacrylate and the terpolymer of poly(diethylaminoethyl methacrylate), of methyl methacrylate and of butyl methacrylate.

According to the invention, the preferred cationic polymer is a terpolymer of poly(dimethylaminoethyl methacrylate), of methyl methacrylate and of butyl methacrylate, such as that sold under the name Eudragit® E by the company Rohm Pharma.

According to the invention, the insoluble cationic polymer(s) preferably represent(s) from 0.5 to 15% by weight relative to the total weight of the pharmaceutical composition, and even more particularly from 1 to 10%.

Moreover, according to a preferred embodiment of the pharmaceutical composition in accordance with the invention, the insoluble cationic polymer/active principle ratio by weight is between 1/5 and 1/30.

The organic or inorganic acid present in the pharmaceutical composition in accordance with the invention may in particular be chosen from citric acid, succinic acid, fumaric acid, acetic acid, phosphoric acid, sulfuric acid and hydrochloric acid.

The use of succinic acid is particularly preferred according to the invention.

This organic or inorganic acid preferably represents from 1 to 10% by weight relative to the total weight of the pharmaceutical composition in accordance with the invention, and even more preferably from 2 to 7% by weight.

The use of an organic or inorganic acid makes it possible to create a micro-pH that promotes the solubilization of the cationic polymer insoluble in water at pH greater than or equal to 5. In fact, the polymer becomes soluble in water by forming a salt in the presence of acid residues.

According to a preferred embodiment of the invention, the organic or inorganic acid/insoluble cationic polymer ratio by weight on which, surprisingly, the dissolution of the active principle also depends, is between 6/1 and 0.25/1, and even more preferably between 2/1 and 0.5/1.

Moreover, the lipophilic active principle/inorganic or organic acid ratio within the pharmaceutical composition in accordance with the invention is preferably between 1/1 and 30/1, and even more preferably it is equal to 20/1.

A particularly preferred pharmaceutical composition according to the invention comprises (by weight relative to the total weight of said composition):
approximately 40 to 80% of fenofibrate as lipophilic active principle,
approximately 2 to 10% of surfactant,
approximately 2 to 10% of a terpolymer of poly(dimethylaminoethyl methacrylate), of methyl methacrylate and of butyl methacrylate (Eudragit E®), and
approximately from 2.5 to 5% of an inorganic or organic acid, the content of approximately 5% being particularly preferred.

Moreover, the pharmaceutical composition that is particularly preferred and as described above can be diluted, if needed, by incorporation of various agents for flow of powders, in a proportion in the region of 40 to 50%.

The pharmaceutical composition in accordance with the invention and as described in general above makes it possible to improve the dissolution of lipophilic active principles that are not very soluble at all, or even insoluble, in water, and in particular that of fenofibrate, and to thus increase their intestinal absorption. The invention therefore provides a pharmaceutical composition comprising a lipophilic active principle that is, in the particular case of fenofibrate, at least 50% dissolved in 15 minutes, more than 80% dissolved in 30 minutes, more than 85% dissolved in 45 minutes and more than 90% dissolved in 60 minutes, as measured in accordance with the method using a paddle rotating at 75 rpm according to the European pharmacopeia, in a dissolving medium consisting of 0.1M sodium lauryl sulfate in aqueous solution brought to 37° C.

Without wishing to be bound to any theory, the improvement in the dissolution of the lipophilic active principles, and in particular in that of fenofibrate, could be facilitated by the acid micro-pH created by the organic or inorganic acid in contact with the cationic polymer insoluble in water, the dissolution of this polymer resulting in better wettability of the surfactant, hence an improvement in the dissolution of the active principle.

The internal phase of the pharmaceutical composition in accordance with the invention can also comprise, according to the needs of the formulation, one or more excipients chosen from diluting agents and/or binders, disintegrating agents and adjuvants conventionally used for spraying, tableting, lubrication and flow of powders known to those skilled in the art, on condition, however, that the presence of such an excipient does not denature the homogeneous and single-phase nature of said internal phase.

Among such excipients, mention may more particularly be made of agents for flow of powders such as talc, silica, stearic acid, magnesium stearate and its derivatives, binders such as celluloses, for instance microcrystalline cellulose, dextrins and maltodextrins, and disintegrating agents such as modified starch, for instance the carboxymethyl starch sold, for example, under the name Primogel®, and their mixtures.

When they are used in the internal phase of the pharmaceutical composition in accordance with the invention, the agents for flow of powders then preferably represent from 40 to 50% by weight, approximately, relative to the total weight of the pharmaceutical composition in accordance with the invention.

The pharmaceutical composition in accordance with the present invention and as described above can be incorporated, among other formulation ingredients, into the formulating of a finished medicinal product. The final pharmaceutical form thus obtained comprises, besides the pharmaceutical composition described above, other components constituting an external phase comprising one or more excipients.

In the context of the present invention, the term “external phase” is intended to mean any coating present on the pharmaceutical composition as described above, on a semi-worked product, on the modified active principle or on an intermediate product. According to the invention, the term “semi-worked product” or “modified active principle” or else “intermediate product” is intended to mean any composition that is intermediate between the starting material consisting of the micronized lipophilic active principle and the finished product. The latter corresponds to the medicinal product in a finished pharmaceutical form, such as a tablet, a gelatin capsule or alternatively a sachet. This semi-worked product may be in various physical forms, in particular an atomized or granulated material, before being incorporated into the final pharmaceutical form.

This external phase may in particular comprise one or more conventional excipients such as those used in the pharmaceutical field and chemically compatible with the active principle used in accordance with the invention.

Among such excipients, mention may in particular be made of diluting agents and/or binders, disintegrating agents, fillers, pigments, wetting agents, buffers, and also one or more adjuvants capable of facilitating the flow of powders, such as flow agents or lubricants (talc, silica, stearic acid, magnesium stearate and its derivatives), commonly used to improve the rheological characteristics of pulverulent products and the final pharmaceutical formulating thereof (packing into bags, dispensing into gelatin capsules, tableting).

Among the binders which may be used in the pharmaceutical composition in accordance with the invention, mention may in particular be made of celluloses such as microcrystalline cellulose, dextrins, maltodextrins, cyclodextrins, mannitol and mixtures thereof. The binders facilitate the tableting operations necessary for the obtaining of tablets and confer an appropriate hardness on the latter. When they are used, these binders preferably represent from 5 to 60% of the total weight of the final pharmaceutical formulation or pharmaceutical form.

Among the disintegrating agents which may be used in the pharmaceutical composition in accordance with the invention, mention may in particular be made of crosslinked sodium carboxymethylcellulose (sodium croscarmellose), modified starch such as the carboxymethyl starch sold, for example, under the name Primogel®, and mixtures thereof.

Among the flow agents or lubricants which may be used in the pharmaceutical composition in accordance with the invention, mention may in particular be made of anhydrous colloidal silica such as the product sold under the name Aerosil®, precipitated silica dioxide, talc, magnesium stearate, stearic acid, polyethylene glycol (PEG), and mixtures thereof; anhydrous colloidal silica being particularly preferred. These flow agents are used to prevent the components of the tablets or the granules from forming aggregates during the preparation of these tablets or granules, and to also reduce the friction during the tableting operation. When they are used, they are generally present in proportions of between 0.1 and 3% of the total weight of the final pharmaceutical form.

The outer phase may also comprise an organic or inorganic acid such as those defined above.

When it is present, the outer phase may represent up to 60% by weight of the total weight of the final pharmaceutical form, preferably up to 45%.

According to a preferred embodiment, the pharmaceutical composition according to the present invention is in the form of tablets.

According to another embodiment, the pharmaceutical composition according to the present invention is in the form of granules dispensed into gelatin capsules.

A subject of the present invention is also a method for preparing such a pharmaceutical composition, comprising the following steps:
a) mixing of at least one solid and micronized lipophilic active principle, of at least one surfactant, of at least one cationic polymer insoluble in water at pH greater than or equal to 5 and of at least one organic or inorganic acid,
b) granulation or atomization of the mixture obtained above in step a),
c) optional addition of an external phase comprising one or more excipients, then
d) tableting or dispensing into gelatin capsules of the mixture obtained at the end of step b) or c) when the latter is carried out.

In step a), the mixture may also optionally comprise an agent for flow of powders such as those described above.

According to a particular embodiment of the invention, the method in accordance with the invention may also comprise a preliminary step of comicronization of the active principle with the surfactant(s).

According to a first variant of the method in accordance with the invention, step b) is carried out by granulation, preferably on a fluidized airbed.

According to this first variant, the mixing of the constituents in step a) preferably comprises the following substeps:
a1) preparing a solution or a suspension comprising at least one organic or inorganic acid and at least one cationic polymer insoluble in water at pH greater than or equal to 5, in a granulating liquid,
a2) spraying the mixture prepared above in step a1) onto the active principle which has been micronized and premixed with the solid surfactant or comicronized with said active principle, at a temperature compatible with the physical stability of the substances used in the formulation, preferably of between 20 and 45° C., and most particularly of between 30 and 40° C.,
a3) recovering the fluidized granules thus obtained,
a4) calibrating, for example by sieving, the fluidized granules, and
a5) drying the fluidized granules.

The granulating liquid used in substep a1) can be chosen from water, organic solvents and mixtures thereof in any proportions, it being possible for said liquid to be optionally added to a buffer specified by the pharmacopeiae in force, said buffer then preferably having a pH of between 2 and 5.

According to a preferred embodiment, the granulating liquid is an organic solvent or a mixture of organic solvents capable of allowing the cationic polymer used to dissolve. By way of such solvents, mention may in particular be made of lower alcohols such as ethanol and propanol, acetone, and mixtures thereof.

When the cationic polymer is a terpolymer of poly(dimethylaminoethyl methacrylate), of methyl methacrylate and of butyl methacrylate (Eudragit® E), then a granulating liquid that is particularly preferred according to the invention is a propanol/acetone mixture, preferably in 60/40 (v/v) proportions.

According to this first variant, the cationic polymer(s) and the inorganic or organic acid used represent, in their entirety, preferably from 10 to 15% by weight of the total weight of the granulating liquid.

When the surfactant is not in the form of a material comicronized with the active principle, said micronized active principle and the solid surfactant used in step a2) above are preferably premixed using a powder mixer such as a tumbling mixer with a fixed or mobile container or a mixer with moving parts such as a planetary mixer, a screw mixer, a ploughshare mixer or a ribbon mixer.

According to a second variant of the method in accordance with the invention, step b) is carried out by atomization.

According to this second variant, the mixing of the constituents in step a) preferably comprising the following substeps:
a′1) preparing an acid or buffer solution comprising an inorganic or organic acid and a strong base, said solution having a pH of less than 5, and preferably of between 2 and 5,
a′2) preparing a suspension by addition, to this buffer solution, of at least one solid and micronized lipophilic active principle, of at least one surfactant optionally comicronized with said active principle and of at least one cationic polymer insoluble in water at pH greater than or equal to 5, with stirring,
a′3) atomizing said suspension,
a′4) recovering the atomized product.

In step a′1), the buffer solution may in particular consist of a mixture of water, of succinic acid and of sodium hydroxide.

Step a′2) is preferably carried out with magnetic stirring and homogenization for 10 to 15 minutes, at a rate of between 8000 and 10 000 rpm. It should, however, be clearly understood that the transposition of scale for an industrial production requires the parameters of duration and of rate of mixing and then of homogenization to be adjusted accordingly, as a function of the equipment used. These parameters will be readily adjusted by those skilled in the art without departing from either the context or the scope of the present invention.

According to a particular embodiment of this second variant, steps a′1) and a′2) can be carried out simultaneously.

In step a′2) and according to a variant of this method, a fraction of the components of the outer phase, when this phase is used, in particular the agents promoting the flow of powders such as starch, silica and/or talc, can be introduced into the suspension to be atomized.

Atomization is a process well known to those skilled in the art which has for a long time been reserved for the agrofoods field. Atomization consists in reducing a body into fine particles, from its liquid state. It involves therefore atomizing a solution or a suspension of one or more solids via a nozzle or another system (turbine, for example) and then evaporating the solvent from the droplets formed.

According to the invention, the atomization of step a′3) is preferably carried out using a two-fluid nozzle, a high pressure nozzle or a turbine, with a flow rate of between 3 and 7 kg/h, preferably approximately 5 kg/h.

The nature of the resulting powder depends on several variables, in particular the initial concentration of the solute, the size distribution of the droplets obtained and the amount of solvent extracted.

In general, the particles of atomized powder are homogeneous, spherical, uniform in size and of low density, with rapid dissolution.

The homogeneity of the size of the particles and their spherical shape gives powders that exhibit good flow and can therefore be directly tableted.

According to the invention, the tableting step d) can, for example, be carried out by dry mixing and direct tableting, in the presence or absence of an external phase as described above.

According to a particularly advantageous embodiment of the invention, a final pharmaceutical form can be prepared according to a method comprising the following steps:
preparing a semi-worked product based on fenofibrate in atomized form, according to the second variant of the method as described above,
mixing the atomized product with an external phase, and
tableting said mixture or dispensing it into gelatin capsules.

The pharmaceutical form thus prepared makes it possible to improve the dissolution of the fenofibrate and to thus increase its intestinal absorption. The invention therefore provides a method for preparing a pharmaceutical composition in the form of an intermediate product dried by atomization, comprising a fenofibrate active principle that is at least 50% dissolved in 15 minutes, more than 80% dissolved in 30 minutes, more than 85% dissolved in 45 minutes and more than 90% dissolved in 60 minutes, as measured in accordance with the method using a paddle rotating at 75 rpm according to the European pharmacopeia, in a dissolving medium consisting of 0.1 M sodium lauryl sulfate in aqueous solution brought to 37° C.

Besides the above provisions, the invention also comprises other provisions which will emerge from the following description, which refers to examples of preparation of pharmaceutical compositions in accordance with the invention based on fenofibrate, and also to an example describing a study comparing the rate of dissolution of pharmaceutical compositions in accordance with the invention with that of the commercially available specialty product Lipanthyl® 160 mg.

It should be clearly understood, however, that these examples are given only by way of illustration of the subject of the invention, of which they in no way constitute a limitation.

### EXAMPLE 1

### Preparation of Pharmaceutical Compositions Based on Fenofibrate by Atomization

A succinate buffer solution, pH 4.6, is prepared by mixing 23.6 g of succinic acid and 164 ml of 1 N sodium hydroxide in a sufficient amount of water to have 2 liters of buffer solution. 8 g of sodium lauryl sulfate (NaLS) are then added to this solution.

400 g of micronized fenofibrate and 40 g of a polymer insoluble in water at pH greater than or equal to 5: cationic copolymer of methacrylic acid having a dimethylaminoethyl group sold under the name Eudragit® E 100 or Eudragit® E PO, are then added, with magnetic stirring and homogenization for 10 minutes at 10 000 rpm using a Turrax® mixer/homogenizer.

A suspension is obtained, which is then atomized in an atomization chamber equipped with a two-fluid nozzle, with a flow rate of 5 kg/h.

The product thus atomized (modified fenofibrate: 195 mg) is then mixed with an external phase, the composition of which is given in table I below:

TABLE I

| | FORMULATIONS | | | | |
|---|---|---|---|---|---|
| Excipients (in mg) | A | B | C | D | E |
| Microcrystalline | 200 | 200 | 200 | 225 | 225 |
| cellulose | | | | | |
| Crospovidone | 42 | — | — | — | — |
| Sodium croscarmellose | — | 42 | 42 | 42 | — |
| Sodium carboxymethyl | — | — | — | — | 42 |
| starch | | | | | |
| PEG 6000 | 3 | 50 | — | — | — |
| Magnesium stearate | 50 | — | 50 | 25 | 25 |
| Anhydrous colloidal | — | 3 | 3 | 3 | 3 |
| silica | | | | | |

The mixture thus obtained is then tableted so as to produce final pharmaceutical formulations A to E in the form of tablets each containing 195 mg of modified fenofibrate corresponding to 160 mg of fenofibrate.

### EXAMPLE 2

### Preparation of a Pharmaceutical Formulation Based on Fenofibrate by Fluidized Airbed Granulation

Using a powder mixer, 8 g of NaLS, 400 g of micronized fenofibrate and 24 g of succinic acid are mixed.

This mixture is then granulated in the fluidized airbed in the presence of a cationic polymer insoluble in water at pH greater than or equal to 5 (Eudragit® E), at 12.5 g in 100 g of a 60/40 (v/v) propanol/acetone mixture.

The granulation product (modified fenofibrate: 195 mg) is then mixed with an external phase in the proportions given below in table II, so as to produce formulation F:

TABLE II

| Excipients (in mg) | Formulation F |
|---|---|
| Microcrystalline cellulose | 225 |
| Sodium carboxymethyl starch | 42 |
| Magnesium stearate | 25 |
| Anhydrous colloidal silica | 3 |

Formulation F is then directly tableted so as to produce tablets F each containing 195 mg of modified fenofibrate, corresponding to 160 mg of fenofibrate.

### EXAMPLE 3

### Comparative Study of the Dissolution Profiles of Formulations A to F Compared With That of Lipanthyl® 160 Mg

The tablets sold under the name Lipanthyl® 160 mg contain 160 mg of micronized fenofibrate and also a mixture of excipients made up of NaLS, lactose monohydrate, polyvidone, microcrystalline cellulose, anhydrous colloidal silica, crospovidone and sodium stearyl fumarate. These tablets correspond to the pharmaceutical composition as also described in patent application FR-A-2 758 459.

This dissolution study consists in measuring the rate of dissolution of the compounds tested according to the method using a paddle rotating at 75 rpm according to the European pharmacopeia (4th edition, paragraph 2.9.3.), in a dissolving medium consisting of 0.1 M NaLS at 37° C.

For each tablet, the dissolution was measured at 15, 30, 45 and 60 minutes.

The results obtained appear in table III below (the values indicated are the means of three measurements of the % dissolution):

TABLE III

TIME (in minutes)

| FORMULATIONS | 15 | 30 | 45 | 60 |
|---|---|---|---|---|
| A | 57 | 84 | 89 | 91 |
| B | 78 | 86 | 89 | 89 |
| C | 78 | 86 | 89 | 90 |
| D | 88 | 95 | 95 | 100 |
| E | 84 | 92 | 94 | 96 |
| F | 83 | 87 | 90 | 93 |
| Lipanthyl ® 160 mg | 75 | 100 | 100 | 100 |

These results show that the formulations in accordance with the invention make it possible to achieve rates of dissolution of at least 50% in 15 minutes, greater than 80% in 30 minutes, greater than 85% in 45 minutes and greater than 90% in 60 minutes.

Consequently, the formulations in accordance with the present invention, while also being much simpler and less expensive to prepare than the compositions described in patent application FR-A-2 758 459, make it possible to achieve dissolution rates that are entirely satisfactory and comparable with that obtained with Lipanthyl® 160 mg.

### EXAMPLE 4

### Preparation of Pharmaceutical Compositions Based on Fenofibrate by Atomization

An acid solution having a pH of between 2 and 5 is prepared by mixing 1 kg of succinic acid in 20 l of water. 3 kg of NaLS are then added to this solution.

In parallel, a solution based on starch and silica is prepared by adding 13 to 15 kg of lipophilic modified starch sold under the name Amidon EmCap® by the company Cerestar and 1 to 2.5 kg of colloidal silica sold under the name Tixosil® by the company Rhodia, to 40 l of water.

15 kg of micronized fenofibrate, 0.5 to 1 kg of an insoluble polymer sold under the name Eudragit® E PO and also approximately 50 kg of the solution based on starch and silica are then added, with magnetic stirring and homogenization for 10 minutes at 10 000 rpm using a planetary mixer.

A suspension is then obtained, which is subsequently atomized as described above in example 1.

The product thus atomized (modified fenofibrate) is then incorporated into the formulation in a proportion of 55 to 65% (i.e. approximately 350 to 400 mg, according to the fenofibrate titer), in the “external phase” mixture, the composition of which is given in table IV below:

TABLE IV

| | FORMULATIONS |
|---|---|
| Excipients (in mg) | G |
| Mannitol | 90 |
| Microcrystalline cellulose | 80-90 |
| Sodium croscarmellose | 42 |
| Magnesium stearate | 2 to 2.5 |
| Anhydrous colloidal silica | 3 to 6.5 |

The mixture thus obtained is then tableted so as to produce a final pharmaceutical formulation G in the form of tablets each containing from 350 to 400 mg of modified fenofibrate, corresponding to 160 mg of fenofibrate.

### EXAMPLE 5

### Comparative Study of the Dissolution Profiles of Formulation G Compared With That of Lipanthyl® 160 Mg

This dissolution study was carried out using the tablets sold under the name Lipanthyl® 160 mg and also composition G prepared above in example 4. The study of the rate of dissolution was carried out according to the method described above in example 3.

For each tablet, the dissolution was measured at 15, 30, 45 and 60 minutes.

The results appear in table V below (the values indicated are the means of three measurements of % dissolution):

TABLE V

TIME (in minutes)

| FORMULATIONS | 15 | 30 | 45 | 60 |
|---|---|---|---|---|
| G | 77 | 98 | 99 | 99 |
| Lipanthyl ® 160 mg | 75 | 100 | 100 | 100 |

As in example 3, these results show that the formulation in accordance with the invention makes it possible to achieve dissolution rates of at least 50% in 15 minutes, greater than 80% in 30 minutes, greater than 85% in 45 minutes and greater than 90% in 60 minutes.

1-32. (canceled)

33. A solid pharmaceutical composition for oral administration; which comprises, within one and the same phase:
a) at least one solid and micronized lipophilic active principle,
b) at least one surfactant,
c) at least one cationic polymer insoluble in water at pH greater than or equal to 5, and
d) at least one organic or inorganic acid.

34. The composition of claim 33, wherein said one and the same phase is an internal phase.

35. The composition of claim 33, wherein the lipophilic active principle comprises blood lipid-reducing compounds, steroid hormones, antifungal compounds, retinoids, steroidal anti-inflammatories, nonsteroidal anti-inflammatories, antiretroviral compounds, protease inhibitors, antacids, proton pump inhibitors, antiemetics, liposoluble vitamins, cardiovascular system drugs, anti-platelet aggregation compounds, anticancer compounds, plant extracts or their isolated or derived active principles, immunosuppressants, central nervous system drugs, antimigraine compounds, antibiotics or antiparasitic compounds or a combination thereof.

36. The composition of claim 35, wherein the blood lipid-reducing compounds comprise 2-(4-(4-chlorobenzoyl)phenoxy)-2-methylpropanoic acid 1-methylethyl ester (fenofibrate), bezafibrate, ciprofibrate, gemfibrozil, probucol, tiadenol, simvastatin, mevastatin, lovastatin, atorvastatin, pravastatin, fluvastatin, cerivastatin or melinamide or a combination thereof.

37. The composition of claim 35, wherein the steroid hormones comprise derived estrogens, esters of estradiol, progesterone, danazol, testosterone, testosterone esters, anti-androgens, 5α-reductase inhibitors, competitive inhibitors of testosterone, quinazoline compounds, nonsteroidal agonists/antagonists of estrogen receptors or a combination thereof.

38. The composition of claim 35, wherein the antifungal compounds comprise itraconazole, miconazole, ketoconazole, fluconazole, griseofulvin, amphotericin B or terbinafine or a combination thereof.

39. The composition of claim 33, wherein the lipophilic active principle is fenofibrate, progesterone or itraconazole.

40. The composition of claim 33, wherein the active principle represents from 10 to 90% by weight of the total weight of the pharmaceutical composition.

41. The composition of claim 33, wherein the surfactant has a hydrophilic-lipophilic balance (HLB) value greater than or equal to 15.

42. The composition of claim 41, the surfactant is selected from the group consisting of sodium lauryl sulfate (HLB 40), poloxamers (HLB 16-29), macrogol ethers of organic alcohols (HLB 15-18), and sucrose esters of organic acids (HLB 15-16).

43. The composition of claim 42, wherein the surfactant is sodium lauryl sulfate.

44. The composition of claim 33, wherein the surfactant represents from 1 to 10% by weight of the total weight of said composition.

45. The composition of claim 33, wherein the surfactant is in solid form.

46. The composition of claim 44, wherein the surfactant is comicronized with the active principle.

47. The composition of claim 33, wherein the active principle/surfactant ratio by weight is between 100/1 and 5/1.

48. The composition of claim 33, wherein the cationic polymers insoluble in water at pH greater than or equal to 5 are acrylic polymers comprising a tertiary amine group.

49. The composition of claim 48, wherein the polymers are aminoalkyl methacrylate polymers, soluble at pH below 5.

50. The composition of claim 48, wherein the polymers are a terpolymer of poly(dimethylaminoethyl methacrylate), of methyl methacrylate and butyl methacrylate; and a terpolymer of poly(diethylaminoethyl methacrylate), methyl methacrylate and butyl methacrylate.

51. The composition of claim 33, wherein the cationic polymer(s) insoluble in water at pH greater than or equal to 5 represent(s) from 0.5 to 15% by weight relative to the total weight of the pharmaceutical composition.

52. The composition of claim 33, wherein the cationic polymer insoluble in water at pH greater than or equal to 5/active principle ratio by weight is between 1/5 and 1/30.

53. The composition of claim 33, wherein the organic or inorganic acid is selected from the group consisting of citric acid, succinic acid, fumaric acid, acetic acid, phosphoric acid, sulfuric acid and hydrochloric acid.

54. The composition of claim 33, wherein the organic or inorganic acid represents from 1 to 10% by weight relative to the total weight of the composition.

55. The composition of claim 33, wherein the organic or inorganic acid/cationic polymer insoluble in water at pH greater than or equal to 5 ratio by weight is between 6/1 and 0.25/1.

56. The composition of claim 33, wherein the lipophilic active principle/inorganic or organic acid ratio is between 1/1 and 30/1.

57. The composition of claim 33, which comprises, by weight relative to the total weight of the composition:
a) 40 to 80% of fenofibrate as lipophilic active principle,
b) 2 to 10% of surfactant,
c) 2 to 10% of a terpolymer of poly(dimethylaminoethyl methacrylate), of methyl methacrylate and of butyl methacrylate, and
d) from 2.5 to 5% of an inorganic or organic acid.

58. The composition of claim 33, which comprises fenofibrate as lipophilic active principle, and wherein it is at least 50% dissolved in 15 minutes, more than 80% dissolved in 30 minutes, more than 85% dissolved in 45 minutes and more than 90% dissolved in 60 minutes, as measured in accordance with the method using a paddle rotating at 75 rpm according to the European Pharmacopeia, in a dissolving medium consisting of 0.1M sodium lauryl sulfate in aqueous solution brought to 37° C.

59. The composition of claim 34, wherein the internal phase comprises one or more excipients comprising diluting agents, binders, disintegrating agents, adjuvants for spraying, tableting, or lubrication and flow of powders or a combination thereof.

60. The composition of claim 34, which further comprises an external phase comprising one or more excipients.

61. The composition of claim 33, which is homogenous.

62. The composition of claim 53, wherein said acid is an organic acid, which is succinic acid.

63. A method for preparing the pharmaceutical composition of claim 33, which method comprises the steps of:
a) mixing of at least one solid and micronized lipophilic active principle, of at least one surfactant, of at least one cationic polymer insoluble in water at pH greater than or equal to 5 and of at least one organic or inorganic acid,
b) granulating or atomizing of the mixture obtained above in step a), then
c) tableting or dispensing into gelatin capsules of the mixture obtained at the end of step b).

64. The method of claim 63, which further comprises after step b), and before step c), adding an external phase comprising one more excipients.

65. The method of claim 63, which further comprises a preliminary step of comicronizing of the active principle with the surfactant(s).

66. The method of claim 63, wherein step b) is carried out by granulation and wherein the mixing of the constituents in step a) comprises the following substeps of:
a1) preparing a solution or a suspension comprising at least one organic or inorganic acid and at least one cationic polymer insoluble in water at pH greater than or equal to 5, in a granulating liquid,
a2) spraying the mixture prepared above in step a1) onto the active principle which has been micronized and premixed with the solid surfactant or comicronized with said active principle, at a temperature compatible with the physical stability of the substances used in the formulation,
a3) recovering the fluidized granules thus obtained,
a4) calibrating the fluidized granules, and
a5) drying the fluidized granules.

67. The method of claim 66, wherein the polymer is a terpolymer of poly(dimethylaminoethyl methacrylate), of methyl methacrylate and of butyl methacrylate and in that the granulating liquid is a propanol/acetone mixture.

68. The method of claim 63, wherein step b) is carried out by atomization and in that step a) comprises the following substeps:
a′1) preparing an acid or buffer solution comprising an inorganic or organic acid and a strong base, said solution having a pH of less than 5,
a′2) preparing a suspension by addition, to this buffer solution, of at least one solid and micronized lipophilic active principle, of at least one surfactant optionally comicronized with said active principle and of at least one cationic polymer insoluble in water at pH greater than or equal to 5, with stirring,
a′3) atomizing said suspension, and
a′4) recovering the atomized product.
